(19) United States
(12) Patent Application Publication
(10) Pub. No.: US 2001/0053862 A1
(43) Pub. Date: Dec. 20, 2001

(54) Process for preparing arylpiperidine carbinol intermediates and derivatives

(76) Inventors: Bruce Ronsen, River Forest, IL (US); Subhash P. Upadhyaya, Chicago, IL (US)

Correspondence Address: OLSON & HIERL, LTD., 36th Floor, 20 North Wacker Drive, Chicago, IL, 60606, US

(21) Appl. No.: 09/814,650

(22) Filed: Mar. 22, 2001

Related U.S. Application Data
(63) Continuation of application No. 09/218,393, filed on Dec. 22, 1998.

(51) Int. Cl. C07C 255/23

(52) U.S. Cl. 558/357

(57) ABSTRACT

A process for the synthesis of arylpiperidine carbinol intermediates and derivatives is disclosed. A preferred process embodiment provides the synthesis of intermediate compounds of structural formula (I) and structural formula (II):

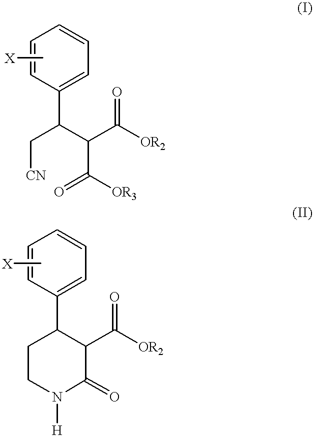

where X is halo, C₁-C₁₀ alkoxy, C₁-C₁₀ haloalkyl, or hydroxy; R₂ and R₃ are each C₁-C₄ alkyl, and R₂ and R₃ are the same. The compound of structural formula (I) is made by condensing a corresponding cinnamonitrile with a corresponding diester malonate. The compound of structural formula (II) in the (±)-trans configuration is obtained by hydrogenating the compound of structural formula (I). The compounds of structural formula (I) and structural formula (II) are useful chemical intermediates for synthesizing 4-arylpiperidine-3-carbinols and their derivatives in (−)-trans configuration.

### CROSS-REFERENCE TO RELATED APPLICATION

This application is a continuation-in-part of co-pending U.S. Ser. No. 09/218,393 filed on Dec. 22, 1998.

### TECHNICAL FIELD

This invention relates to arylpiperidine carbinol intermediates and derivatives, as well as methods for their preparation.

### BACKGROUND OF THE INVENTION

The preparation of pharmacologically active arylpiperidine derivatives by conversion of the primary hydroxyl residue of the carbinol on the arylpiperidine into an ether with either an aliphatic and/or aromatic residue has been described in U.S. Pat. No. 4,007,196 by Christensen et al., and in U.S. Pat. No. 4,902,801 by Faruk, et al., and by others. Further, derivatives of the secondary amine of the piperidine residue can have significance both biologically and chemically.

The arylpiperidine carbinols can be represented by the following general structural formula (1), which can be derivatized by substituents on the heterocyclic nitrogen atom, on the aromatic ring, as well as for the hydrogen of the hydroxymethyl group.

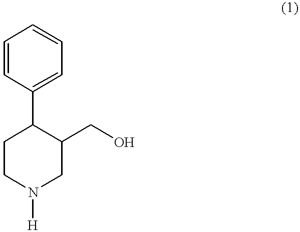

Of particular interest is paroxetine, i.e., (−)-trans-(4R,3S)-4-(p-fluorophenyl)-3-[[3,4-(methylenedioxy)phenoxy]methyl]-piperidine. Its hydrochloride salt (paroxetine HCl), preferably in an amorphous form as described in U.S. Pat. No. 5,672,612 by Ronsen and El-Rashidy, has been shown to be pharmacologically active. Paroxetine is useful in managing diseases of the central nervous system. In particular, depression, obsessive compulsive disorder, PMS (premenstrual syndrome), social anxiety disorder, and the like. Further, paroxetine has been found to be of particular benefit in treating premature ejaculation, a sexual performance condition affecting men. See, for example, U.S. Pat. No. 5,276,042, to Crenshaw et al.

The pharmacological properties of the substituted arylpiperidine carbinols are primarily expressed by a specific stereochemical arrangement of the substituents of the various residues. Most organic molecules have atoms arranged in a three dimensional array. When the same number and kind of atoms are arranged in different ways, the resulting compounds are referred to as isomers. A carbon atom bonded to four different substituents (i.e., a “chiral center”) can have two mirror image arrangements of those substituents; this is a special type of isomerism referred to as stereoisomerism.

One property of chiral centers is that they can rotate plane polarized light, and thus chiral centers are also referred to as optical centers. The two mirror images rotate plane polarized light to exactly the same extent, but in opposite directions. Each chiral center in a molecule contributes to the overall optical rotation of that molecule. Molecules without chiral centers do not rotate plane polarized light.

Often, organic molecules have more than one chiral center, each having two potential stereoisomers. When two molecules, having one or more chiral centers, have all of their atoms arranged in the exact mirror image of the other (i.e., each chiral center in one molecule is the mirror image of each chiral center in the other molecule), these isomers are “enantiomers”, or “optical isomers.” Two otherwise identical molecules, having two or more chiral centers, may be arranged with some chiral centers as mirror images of the corresponding centers in the other molecule and some chiral centers in an identical arrangement. Such molecules are “diastereomers” of each other. The arrangement of groups around a chiral center is referred to as “absolute stereochemistry.” The relationship between different chiral centers in a molecule is “relative sterochemistry.”

A substantially equal mixture of two enantiomers is referred is a “racemic mixture” or “racemate.” Racemic mixtures do not rotate plane polarized light, since each enantiomer affords an equal and opposite rotation.

The International Union of Pure and Applied Chemistry (IUPAC) has adopted a nomenclature for describing the absolute sterochemistry of a chiral center, known as the Cahn-Ingold-Prelog (C-I-P) convention. Under the C-I-P convention, each chiral center is given a designation of R or S. For a given chiral center, the R and S configurations are mirror images. The basic rules for determining whether a center is R or S are described in most organic chemistry texts, such as Carey & Sundberg, Advanced Organic Chemistry, Part A, Plenum Press, New York (1977).

Only the (−)-trans-(4R,3S) substituted configuration of arylpiperidine carbinols lead to drugs with the desired pharmacodynamic properties. This requires the synthesis and purification of the selected desired enantiomer of the arylpiperidine carbinol. A particularly desired enantiomer precursor for preparing pharmacologically active arylpiperidine carbinols is the 4-arylpiperidine-3-carbinol in (−)-trans-(4R,3S) configuration.

The 4-arylpiperidine-3-carbinols exist in four stereoisomers since there are two chiral centers in this molecule, i.e. trans (4R,3S); trans (4S,3R); cis (4R,3R); and cis( 4S,3S). The two trans isomers are enantiomers of one another and will have equal and opposite optical rotations. Likewise, the two cis isomers are enantiomers of each other, whereas the cis and trans forms are related to each other as diasteromers.

Multi-substituted cyclic compounds such as the arylpiperidines shown below, exhibit relative stereochemistry where two or more substituents are either on the same face of the ring (cis) or on opposite faces of the ring (trans). In the structural formulas below, solid-wedged bonds project above the plane of the ring and dashed-wedged bonds project below the plane of the ring. Thus, the upper compounds in the structures having a phenyl substituent projecting above the plane and a hydroxymethyl substituent projecting below the plane of the ring represent trans stereochemistry.

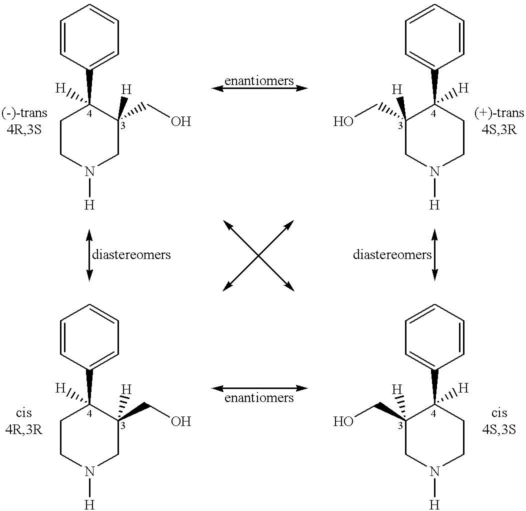

For reactions involving non-optically active intermediates (i.e. achiral or racemic intermediates and reagents) racemic products will inevitably result. When reagents and/or intermediates are chiral (optically active) chemical reactions may result in products that are enantiomerically pure or enriched. Alternatively, racemic materials may be separated into their pure enantiomers by processes commonly referred to as “resolutions,” which utilize chiral media or reversible chemical reactions with chiral reagents to effect the separation. Normally, drugs must be manufactured in their enantiomerically pure form. Thus, for drug manufacturing purposes, the synthesis of the appropriate precursor requires reactions and purification steps favoring the desired trans relationship between the two chiral centers (relative stereochemistry) and a resolution or other chiral induction step to produce the necessary absolute stereochemistry of (−) trans (4R,3S).

Lambrecht et al., in Arch. Pharm., 308, 676 (1975), describe the synthesis of racemic cis/trans 4-arylpiperidine-3-carbinols by the Grignard reaction shown generally in Synthesis Scheme A.

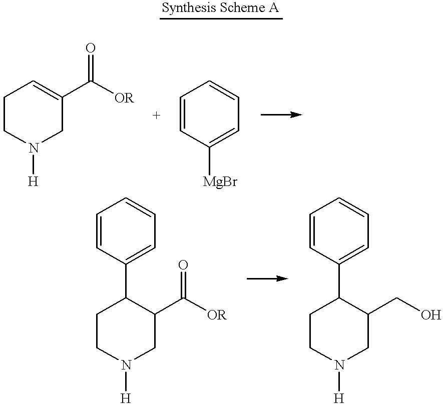

wherein R can be an alkyl group or the like.

Alternatively, several other methods of preparing arylpiperidine carbinols have been disclosed. For example, U.S. Pat. No. 4,902,801 to Faruk, et al., describes in situ cyclization of the branched derivative prepared by the reaction of fluorobenzaldehyde with ethyl acetate followed by Michael addition of amidomalonate to the adduct; and U.S. Pat. No. 4,861,893 to Borrett, et al. describes the derivativization of pyridine followed by the reduction of the aromatic pyridine to the piperidine. However, the foregoing methods described do not account for the four stereoisomers so formed and tend to produce less than optimum yields of (−)-trans (4R,3S) isomer because the (+)-cis, (−)-cis, and the (+)-trans isomers also all form.

Due to the difference in free energies between cis and trans forms of substituted arylpiperidines, certain compounds form predominantly in the trans configuration during the piperidine ring formation process, depending on the substitution pattern of the acyclic precursor molecule. For example, Faruk et al. obtained trans-4-(4′-fluorophenyl)-3-ethoxycarbonyl-N-methylpiperidine-2,6-dione via in situ cyclization of 1-(4′-fluorophenyl)-1-(ethoxycarbonylmethylene)-N-methylamidomalonate. In contrast, Wang et al. in European Patent Application EP 0802185 A1, obtained a mixture of cis and trans-4-(4′-fluorophenyl)-3-ethoxycarbonyl-piperidin-6-one from the cyclization of diethyl 2-cyano-3-(4′-fluorophenyl)glutarate. From these examples, it appear that an open chain piperidine precursor having a geminial dicarbonyl substitution favors formation of trans 4-phenyl-3-alkoxycarbonyl substituted piperidines, whereas replacement of one of the geminal dicarbonyl substituents by a cyano group leads to a cis/trans mixture (see Synthesis Scheme B).

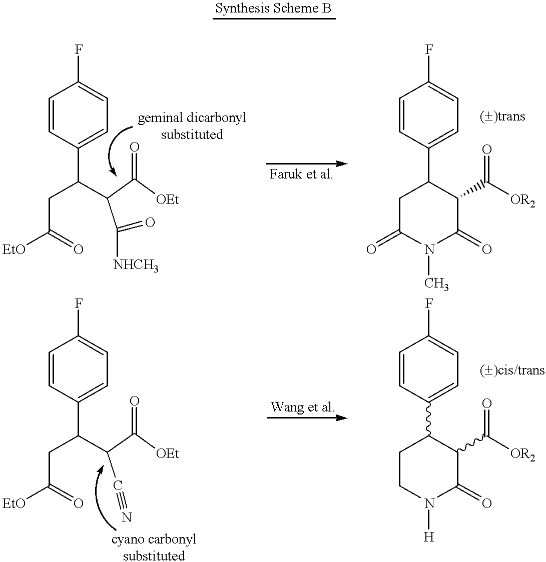

Koelsch, in J. Am. Chem. Soc., 65, 2459 (1943), describes the synthesis of 3-ethoxycarbonyl-4-phenylpiperidin-6-one (II) and its 2-keto positional isomer 3-ethoxycarbonyl-4-phenylpiperidin-2-one (V) by the routes shown in Synthesis Scheme C.

Reductive cyclization of diethyl-2-cyano-3-phenylglutarate (I) with hydorgen and Raney nickel catalyst at 2000 pounds of hydrogen pressure produced 3-ethoxycarbonyl-4-phenylpiperidin-6-one (II) in 67% yield. Reduction of (II) with sodium metal in butanol resulted in reduction of only the 6-keto group and the hydrolysis of the ester group to afford 4-phenylpiperadine-3-carboxylic acid (III) in a modest 41% yield.

Interestingly, reductive cyclization diethyl 1-[1-(cyanomethyl)-1-(phenyl)methyl]-malonate (IV) e.g., a positional isomer of (I), with hydogen and Raney nickel, under the same conditions as cyclization of (I) above, afforded an undisclosed yield of 3-ethoxycarbonyl-4-phenylpiperidin-2-one (V) as a syrup, and a 28% isolated yield of crystalline 4-phenylpiperidin-2-one (VI). Compound (VI) is formed by decarbonylation of either the starting compound (IV) or of product compound (V). It is fairly common for positional isomers such as compounds (I) and (IV) to react differently under the same reaction conditions. The relative positions of various substituents in molecules can have profound effects on the reactivity of the individual substituents due to electronic and steric influences. The isolation, purification and reduction of (V) are not disclosed by Koelsch.

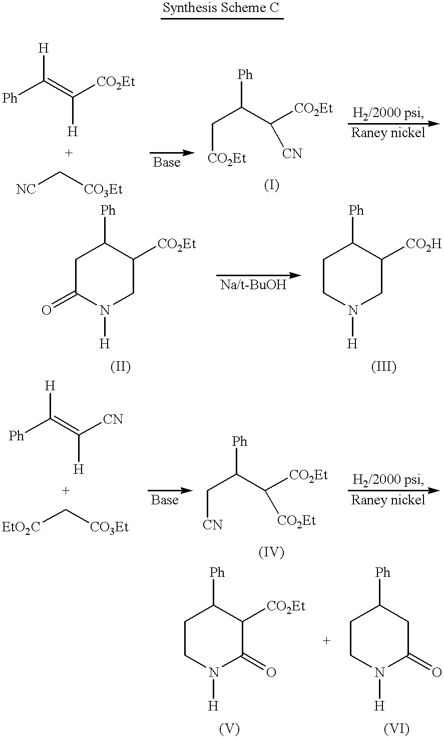

Wang et al., in European Patent Application EP 0802185 A1, describe the preparation of 3-alkoxycarbonyl-4-arylpiperidin-6-ones similar to compound (II) of Koelsch. Wang et al. synthesize the 6-keto-4-arylpiperidine via Michael addition of ethyl cyanoacetate to ethyl 4-fluorophenylcinnamate followed by Raney nickel hydrogenation, under pressure, in a manner similar to Koelsch's synthesis of compound (II) in Synthesis Scheme B. The reductive cylization afforded 3-ethoxycarbonyl-4-(4-fluorophenyl)piperidin-6-one as a racemic cis-trans mixture under most of the reaction conditions reported. Wang et al. also disclose metal hydride reduction of the 6-keto group to a methylene and the alkoxycarbonyl group to a hydroxymethyl group.

The present invention provides a process for the synthesis of arylpiperidine carbinols in the optically active (−)-trans-(4R,3S) configuration and intermediates in the racemic trans configuration for synthesizing arylpiperidine carbinols, without the necessity if removing or isomerizing cis products, and utilizing inexpensive commercially available malonate diesters.

### SUMMARY OF THE INVENTION

A process for the synthesis of arylpiperidine carbinol intermediates and derivatives is disclosed. In particular, the inventive process provides for the synthesis of racemic (±)-trans intermediate compounds and derivatives, which are useful precursors for a simplified synthesis of arylpiperidine carbinols in optically pure (−)-trans configuration.

The inventive process synthesizes novel intermediate compounds having structural formula (I) and structural formula (II):

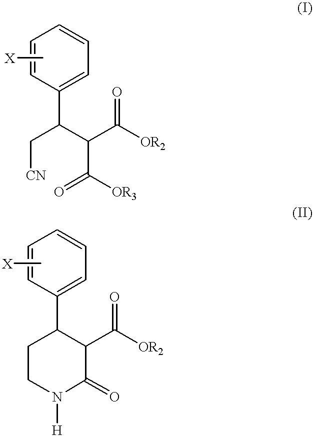

where X is halo, C₁-C₁₀ alkoxy, C₁-C₁₀ haloalkyl, or hydroxy, and each of R₂ and R₃ is C₁-C₄ alkyl and R₂ and R₃ are the same.

In preferred process embodiments, compounds of structural formula (I) can be synthesized by condensing a cinnamonitrile of structural formula (III), wherein X is as defined in structural formula (I) with a diester malonate of structural formula (IV), wherein each of R₂ and R₃ is as defined in structural formula (I).

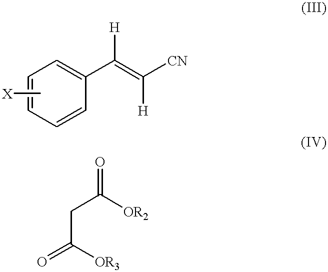

In another preferred process embodiment, compounds of structural formula (II) can be synthesized by hydrogenating compounds of structural formula (I).

A preferred compound of structural formula (I), diethyl-[1-cyanomethyl-1-(4′-fluorophenyl)methyl]-malonate, can be synthesized in the form of a substantially pure, racemic crystalline solid having a melting point temperature in the range of about 35° to about 50° C., preferably in the range of about 45° to about 48° C.

The compounds of structural formula (I) and structural formula (II) are useful racemic chemical intermediates for the synthesis of 4-arylpiperidine-3-carbinols in (−)-trans-(4R,3S) configuration.

The inventive process advantageously provides compounds of structural formula (II) as a racemic, predominantly (±)-trans configured mixture, thereby avoiding the need to remove or isomerize any minor amounts of cis configured diasteriomer compounds that form. Thus, in this reaction, substantially only two of the four possible stereoisomers form (e.g., the enantiomeric trans isomers) due to the kinetic and thermodynamic conditions of the reaction and the favorable substitution pattern of the starting Compound (I).

In one preferred process embodiment, a racemic intermediate compound of structural formula (II), (±)-trans-4-(4′-fluorophenyl)-3-ethoxycarbonyl-piperidin-2-one was synthesized by hydrogenation of (I), which, upon further reduction, produced racemic (±)-trans-4-(4′-fluorophenyl)piperidine-3-carbinol. Beneficially, 4-(4′-fluorophenyl)-3-ethoxycarbonyl-piperidin-2-one can be synthesized in the form of a substantially pure, racemic crystalline solid having a melting point in the range of about 140° to about 150° C. and substantially (±)-trans configuration (i.e. greater than 90% trans). Thus, this inventive process avoids the need for additional workup to remove cis configured diastereomer compounds and simplifies the process for purification of the inventive intermediate of structural formula (II) to the desired biologically active (−)-trans (4R,3S)-4-arylpiperidine-3-carbinols.

### DETAILED DESCRIPTION OF PREFERRED EMBODIMENTS

As used herein, the term “alkyl” includes both branched and straight-chain saturated aliphatic hydrocarbons; and the term “haloalkyl” means that the alkyl group is as defined above and substituted with one or more halogen atoms. The term “halo” as used herein refers to fluoro, chloro, bromo, or iodo. The term “aryl” as used herein, refers to a carbocyclic aromatic moiety, such as phenyl, benzyl, naphthyl, and the like. The term “alkali metal” refers to sodium, potassium, lithium and the like.

The inventive process is particularly well suited for the synthesis of 4-arylpiperidine-3-carbinols in (−)-trans-(4R,3S) configuration and racemic intermediates for the synthesis thereof.

One process embodiment of this invention is illustrated generally by Synthesis Scheme (1), below which comprises reacting a substituted benzaldehyde (Compound A) with acetonitrile in the presence of alkali metal hydroxide to form a cinnamonitrile (Compound B); condensing Compound B with dialkyl malonate in a basic medium, containing a solvent and a base, preferably an alkali metal alkoxide base, and preferably an alkyl ester solvent, to form a diester intermediate of structural formula (I) (Compound C). Preferably the alkyl group of each of the dialkyl malonate, alkali metal alkoxide and alkyl ester is the same to avoid the formation of mixed ester groups in the intermediate compound of structural formula (I).

Also illustrated in Synthesis Scheme (1) is a further process embodiment of this invention, which comprises hydrogenating Compound C to a racemic (±)-trans monoester piperidin-2-one intermediate compound of structural formula (II) (Compound D); and further process embodiments of reducing Compound D with a metal hydride such as lithium aluminum hydride, aluminum hydride, and the like, to racemic (±)-trans arylpiperidine base (Compound E); alkylating Compound E to the racemic (±)-trans N-substituted compound (Compound F); and isolating from Compound F the substantially enantiomerically pure (−)-trans configured arylpiperidine carbinol (Compound G). Aluminum hydride may be produced in situ by treatment of lithium aluminum hydride with a mineral acid such as sulfuric acid.

Compound G can be isolated by resolving Compound F in two steps as illustrated in Synthesis Scheme (1). In Step 1, Compound F is dissolved in a suitable solvent, preferably acetone. To the resulting solution is added a solution of appropriate chiral acid, e.g., (−)-Di-p-toluoyl tartaric acid or other tartaric acid, or derivative thereof, dissolved in the same or an appropriate solvent to form a salt. The salt so formed with (−)-trans-arylpiperidine carbinol crystallizes, while the salt formed with the (+)-trans-compound remains in solution. In Step 2, the crystallized salt is neutralized with aqueous base, preferably potassium hydroxide, to afford the substantially enantiomerically pure (−)-trans-arylpiperidine carbinol (Compound G). Compound G can then be readily recovered and purified.

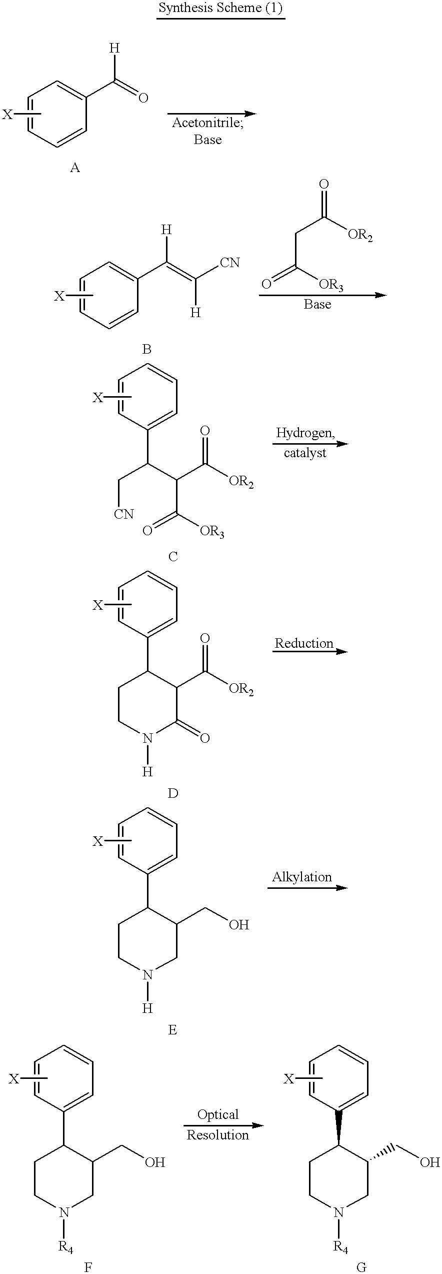

In general Synthesis Scheme (1): X in each of Compounds A, B, C, D, E, F, and G is halo, C₁-C₁₀ alkoxy, C₁-C₁₀ haloalkyl, or hydroxy; each of R₂ and R₃ is C₁-C₄ alkyl and R₂ and R₃ are the same; and in each of Compounds F and G, R₄ is C₁-C₁₀ alkyl.

The inventive intermediates and inventive process greatly simplify the preparation from Compound E to the desired biologically active (−)-trans arylpiperidine carbinol compound and derivatives thereof, and 4-arylpiperidine-3-carbinols in particular.

In a preferred process embodiment, Compound A is 4-fluorobenzaldehyde, the alkali metal hydroxide is potassium hydroxide, the diester malonate is diethyl malonate and the base and solvent medium comprises sodium ethoxide and ethyl acetate, respectively, and Compound F is methyl-N-substituted as illustrated generally in Synthesis Scheme (2). Compound G can be prepared as described above.

Advantageously, Compound C is provided in Synthesis Scheme (2) as diethyl-[1-cyanomethyl-1-(4′-fluorophenyl)methyl]-malonate and recoverable in the form of a substantially pure, racemic crystalline solid having a melting point in the range of about 35° to about 50° C., preferably in the range of about 45° to about 48° C.

Beneficially, Compound D is provided in Synthesis Scheme (2) as substantially (±)-trans 4-(4′-fluorophenyl)-3-ethoxycarbonyl-piperidin-2-one, which upon reduction produces (±)-trans 4-(4′-fluorophenyl)piperidine-3-carbinol (Compound E). Advantageously, Compound D is also recoverable in the form of a substantially pure, racemic crystalline solid having a distinct melting point temperature in the range of about 140° to about 150° C. Further advantages of this process are that Compound D so formed has substantially the (±)-trans configuration, and thus requires no additional steps to remove or isomerize the cis configured material, small amounts of which do not interfere with the subsequent reactions.

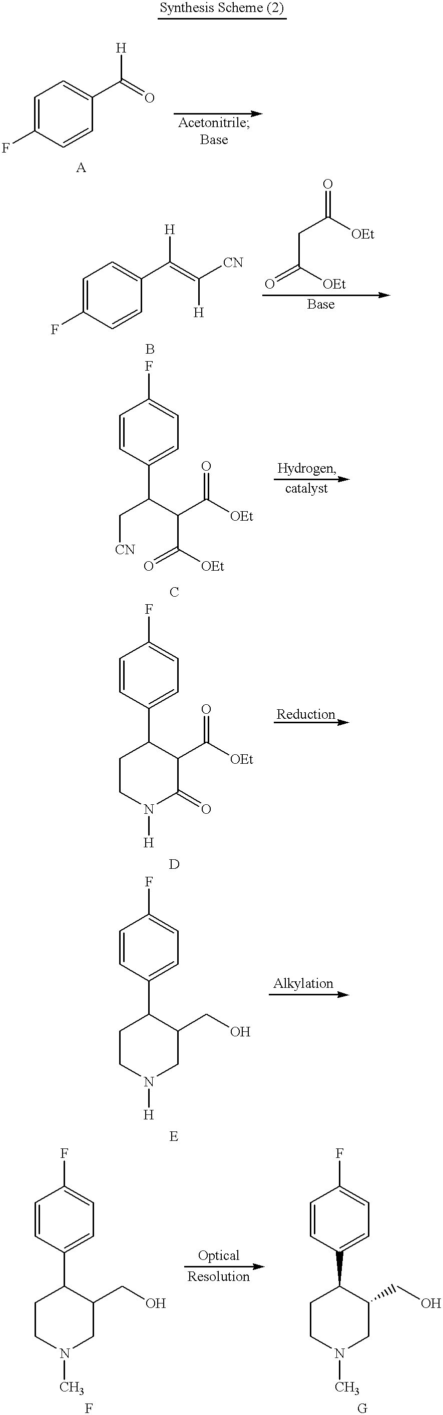

The following examples illustrate preferred embodiments of the preparation and characterization of the inventive intermediates and inventive process prepared by Synthesis Scheme 2 without limitation thereto.

### EXAMPLE 1

### Synthesis of 4-Fluorocinnamonitrile (Compound B)

Powdered KOH (13.5 g, 85%) was suspended in acetonitrile (100 mL) solvent and mixed with stirring in a water bath at a temperature in the range of about 45° to about 50° C. 4-Fluorobenzaldehyde (20 g) (Compound A) was dissolved in acetonitrile (30 mL) solvent and the resulting solution was added in a stream to the stirred mixture. The resulting reaction mixture was further stirred at the foregoing temperature for about 30 minutes after which the reaction mixture was quenched by pouring it into a beaker containing crushed ice (130 g). An upper organic layer separated and was washed with brine (50 mL), dried over sodium sulfate and the solvent was evaporated under reduced pressure to provide a substantially semi-solid crude material (17.8 g). The crude material was passed through silica gel (30 g) using 25% ethyl acetate in hexane to provide the title Compound (B) in the form of a pale yellow semi-solid product (16.7 g, 70% yield, trans/cis or E/Z ratio about 4 by NMR). Analysis by High Performance Liquid Chromatography (HPLC) showed it to be >95% pure. Compound (B) was characterized as follows.

¹H-NMR (CDCl₃): trans-B, δ 5.83 (d, 1H, J=16.8 Hz), 7.00-7.90 (m,5H); cis-B, δ 5.48 (d, 1H, J=12.3 Hz), 7.00-7.90 (m, 5H).

Mass Spectra: Mass Spectra (CI, Methane); m/e (relative intensity): 148 (M⁺+1 m 100), 272 (79), 254 (56), 125 (10), 109 (34). Analysis: Calculated for C₉H₆FN: C, 73.46; H, 4.11; N, 9.52. Found: C, 72.65; H, 4.24; N, 9.04.

A small sample (0.22 g) was rechromatographed on silica gel (0.5 g) using 5% ethyl acetate in hexane (10 mL) to give white crystalline solid (0.16 g). Analysis: Calculated for C₉H₆FN: C, 73.46; H, 4.11; N, 9.52. Found: C, 73.44; H, 4.04; N 9.32.

A description of another process for the synthesis of 4-fluorocinnamonitrile can be found in DiBiase, S. A. et al., J. Organic Chemistry, 44, 4640-4649 (1979), the relevant disclosures of which are incorporated herein by reference (hereafter the “DiBiase Process”). However, the DiBiase et al. process employs relatively higher temperatures (reflux), a further extraction of the product with dichloromethane, drying over sodium sulfate and evaporation in vacuo at a bath temperature of 30° C. and produced only a 50% yield. Therefore, the foregoing procedure was found to be an improvement over the DiBiase process, because it minimizes cinnamonitrile reaction in situ due to the relatively lower temperature employed and affords easier workup of the product.

### EXAMPLE 2

### Synthesis of Diethyl[1-cyanomethyl-1-(4′-fluorophenyl)methyl]malonate (Compound C)

Compound B was prepared by the process described in Example 1. Sodium ethoxide (0.7 g) base was added to a solution of Compound B (1.47 g) dissolved in ethyl acetate (15 mL) solvent at ambient room temperature. Diethyl malonate (1.8 g) was added to the solution. The resulting reaction mixture was stirred overnight at ambient room temperature and then refluxed for 4 hours. The reaction was determined to be incomplete, based on analysis by Thin Layer Chromatography (TLC) (R_{f} values of 4-fluorocinnamonitrile=0.58; of diethyl malonate=0.54; and of reaction product=0.41), employing a solvent system of hexane and ethyl acetate (7:3), ultraviolet light and iodine vapor to expose the spots. Silica gel fluorescent plates were used.

Therefore, more sodium ethoxide (0.54 g) was added and the reaction mixture was refluxed further for about 2 hours. Completion of the reaction was determined by TLC as described above. The reaction mixture was then cooled to ambient room temperature and quenched with a solution of glacial acetic acid (1.5 g) in water (10 mL). Ethyl acetate (10 mL) was then added and an upper organic layer separated.

The organic layer was washed with brine (10 mL), dried over sodium sulfate and the solvent was evaporated under reduced pressure to provide 3.2 g crude product in the form of a dark yellow oil. This crude oil product (3.0 g) was purified by flash column chromatography using silica gel (20 g, 230-400 mesh) using hexane and then 1-2% ethyl acetate in hexane to provide the title Compound C (2.07 g, 72% yield) in the form of a substantially colorless oil, which crystallized on standing, and was recoverable as an off-white solid having a melting point in the range of about 45° to about 48° C. Compound C was analyzed as having the following spectral characteristics.

¹H-NMR (CDCl₃): δ 1.03 (t, J=7.2 Hz, 3H), 1.30 (t, J=7.2 Hz, 3H), 2.88 (d, J=7.2 Hz, 2H), 3.75 (m, 1H), 3.83 (d, J=10.2, 1H), 3.98 (q, J=7.2 Hz, 2H), 4.26 (q, J=7.2 Hz, 2H), 7.00-7.35 (m, 4H).

¹³C-NMR (CDCl₃): δ 13.6, 13.8, 22.7, 40.4, 55.8, 61.7, 62.1, 115.8 (d, J=14.7 Hz), 117.4, 129.5, 133.7, 161.8 (d), 166.7, 167.3.

Mass Spectra (EI); m/e (relative intensity): 307 (M⁺, 45), 262 (12), 234 (90), 216 (33), 205 (78), 149 (96), 148 (100). Mass Spectra (CI, Methane); m/e (relative intensity): 308 (M⁺+1, 100), 262 (43), 234 (6), 216 (14).

Analysis: Calculated for C₁₆H₁₈FNO₄: C, 62.53; H, 5.90; F, 6.18; N, 4.56. Found: C, 62.43; H, 6.04; F, 6.32; N, 4.22.

### EXAMPLE 3

### (±)-trans-3-Ethoxycarbonyl-4-(4′-fluorophenyl)piperidin-2-one (Compound D)

Compound C of Example 2 (1.5 g) was dissolved in methanol (30 mL) and to the solution was added activated Raney nickel (1.45 g, 50% wet) catalyst. The reaction mixture was purged with nitrogen and then stirred under hydrogen atmosphere (H₂) at atmospheric pressure in a water bath set at a temperature of about 40° C. for a period of about 16 hours. The reaction mixture was then cooled to ambient room temperature and TLC analysis was performed as described in Example 2. TLC analysis showed that the reaction was complete (R_{f} values of starting material=0.41; of reaction product=0.12).

The cooled reaction mixture was filtered through Celite. The reaction flask and Celite residue were each washed with methanol (15 mL) and the filtrates combined.

The combined filtrate was evaporated under reduced pressure to provide a pale yellow solid crude product (1.2 g). This crude product was suspended in a 4:1 mixture of hexane and ethyl acetate (10 mL) and the mixture was stirred at about 5° C. for about 1 hour, and then filtered and air dried to give the title Compound D (0.91 g, 70% yield) recovered in the form of a crystalline white powder, having a melting point in the range of about 145° to about 147° C. Compound D was analyzed as having the following spectral characteristics.

¹H-NMR (CDCl₃): δ 1.08 (t, J=7.2 Hz, 3H), 2.05 (m, 2H), 3.35-3.60 (m, 4H), 4.09 (q, J=7.2 Hz, 2H), 6.89 (br. S. 1H), 6.95-7.25 (m, 4H).

¹³C-NMR (CDCl₃): δ 13.8, 29.0, 41.2, 41.6, 56.2, 61.1, 115.5 (d, J=10.2 Hz), 128.4, 137.2, 161.8 (d), 168.3, 169.5

No resonances characteristic of the cis isomer were observed in either the proton or carbon NMR spectrum.

Mass Spectra (EI): m/e (relative intensity): 265 (M³⁰, 3), 220 (5), 192 (100), 163 (4), 162 (3), 149 (13). Mass Spectra (CI, Methane); m/e (relative intensity): 266 (M³⁰+1, 100), 246 (4), 220 (18), 294 (7), 192 (9);

Analysis: Calculated for C₁₄H₁₆FNO₃: C, 63.39; H, 6.08; F, 7.16; N, 5.28. Found: C, 63.28; H, 6.26; F, 7.28; N, 5.14.

### EXAMPLE 4

### Diethyl[1-cyanomethyl-1-(4′-fluorophenyl)methyl]malonate (Compound C)

Compound B of Example 1 (10 g) was dissolved in ethyl acetate (100 mL) and diethyl malonate (10.88 g) was added to the solution. The resulting mixture was stirred under nitrogen atmosphere at ambient room temperature. Sodium ethoxide (7.5 g) was slowly added to the mixture. After the addition was complete, the resulting combination of reactants was refluxed for about 1.5 hours. The produced reflux mixture was then cooled to ambient room temperature. TLC analysis was performed as described in Example 2 and showed the presence of starting material, so more sodium ethoxide (2.5 g) was added and the mixture was further refluxed for about 3.5 hours. TLC analysis showed that the reaction was complete.

The reaction mixture was then cooled to ambient room temperature and the procedure for obtaining Compound C from the cooled reaction mixture as described in Example 2 was followed, except that no acetic acid was employed. The title Compound C was provided in the form of a light yellow oil, which on standing, crystallized recovered as an off-white solid having a melting point in the range of about 44° to about 46° C. (15.2 g, 73% yield). The chromatography data and spectral ¹H-NMR data were substantially the same as the data obtained for Compound C prepared in Example 2.

### EXAMPLE 5

### (±)-trans-3-Ethoxycarbonyl-4-(4′-fluorophenyl)piperidin-2-one (Compound D)

Compound C of Example 3 (12.0 g) was reacted with activated Raney nickel (9.4 g, 50% wet) catalyst in methanol (150 mL), following the catalytic hydrogenation procedure of Example 3 except that a reaction period of about 48 hours (weekend) was employed. The title Compound D was recovered in the form of a crystalline white solid (9.0 g, 87% yield) having a melting point of 141-142° C. The TLC and HPLC results for Compound D were similar to those of Compound D prepared in Example 3.

Analysis: Calculated for C₁₄H₁₆FNO₃: C, 63.39; H, 6.08; F, 7.16; N, 5.28. Found: C, 63.34; H, 6.16; N, 5.22.

### EXAMPLE 6

### (±)-trans-4-(4′-Fluorophenyl)-3-hydroxymethylpiperidine (Compound E)

Compound D of Example 5, (4.4 g) was suspended in tetrahydrofuran (30 mL) and then added slowly to a slurry of lithium aluminum hydride (LiAlH₄) (1.5 g) in anhydrous tetrahydrofuran (30 mL) while concurrently cooling in an ice-water bath. The reaction mixture was then refluxed for about six hours under a nitrogen atmosphere. The reflux mixture was then cooled to ambient room temperature. The cooled mixture was then quenched by concurrently adding water (10 mL) slowly while further cooling the mixture in an ice water bath set at a temperature of about zero to about 4° C., followed by the addition of 10% aqueous sodium hydroxide (2 mL) at about ambient room temperature. The resultant slurry was stirred for about one hour and then the aluminum hydroxide containing solids were filtered and the reaction vessel and solids were washed with ethyl acetate (50 mL) and the filtrates combined. The combined filtrate was dried over sodium sulfate and the solvent was evaporated under reduced pressure to an oily residue. The oily residue was dissolved in ethyl acetate (25 mL) and allowed to stand undisturbed overnight at ambient room temperature. A solid product separated which was then filtered and washed with cold (zero to about 5° C.) ethyl acetate (10 mL). The title Compound E (0.80 g, 25% yield) was recovered as a crystalline white powder having a melting point of 123-124° C. Compound E was analyzed as having the following spectral characteristics.

¹H-NMR (CDCl₃): δ 1.56-1.92 (m, 5H), 2.58 (t. 1H), 2.71 (m, 1H), 3.16 (d, 1H), 3.24 (dd, 1H), 3.38 (m, 1H), 6.98 (m, 2H), 7.17 (m, 2H).

Mass Spectra (CI, Methane); m/e (relative intensity): 210 (M³⁰+1, 100), 202 (24), 192 (54), 178 (3), 126 (84);

Analysis: Calculated for C₁₂H₁₆FNO: C, 68.88; H, 7.71; F, 9.08; N, 6.69. Found: C, 68.47; H, 7.74; F, 8.99; N, 6.59.

### EXAMPLE 7

### (±)-trans-4-(4′-Fluorophenyl)-3-hydroxymethyl-N-methylpiperidine (Compound F)

Compound E of Example 6, (0.37 g) was dissolved in methanol (10 mL). Activated Raney nickel (0.30 g, 50% wet) catalyst and aqueous formaldehyde (formalin) solution (0.30 g, 37 wt %) were added to the solution. The reaction mixture was stirred at a temperature of about 50° C. under hydrogen (H₂) at atmospheric pressure for about 17 hours. The mixture was diluted with methanol (10 mL) and filtered through celite (1 g). The filtrate was evaporated under reduced pressure to give an oil (0.41 g). This oil was stirred with hexane (10 mL) overnight. A solid separated which was filtered and washed with hexane (5 mL) to give the title Compound (F) as a white solid (0.33 g, 84% yield), with a melting point of 117-120° C. Compound F was characterized as follows.

¹H-NMR (CDCl₃): δ 1.70-2.15 (m,5H), 2.20-2.45 (m,4H), 2.60-2.80 (m,1H), 2.90-3.00 (d,1H), 3.10-3.30 (m,2H), 3.35-3.45 (dd,1H), 6.95-7.25 (m,4H) ppm.

Mass Spectra (CI, Methane); m/e (relative intensity): 224 (100), 206 (93), 179 (3), 128 (7).

Analysis: Calculated for C₁₃H₁₈FNO: C, 69.93; H, 8.13; F, 8.51; N, 6.27. Found: C, 69.37; H, 8.11; F, 8.59; N, 6.16.

### EXAMPLE 8

### (−)-trans-4-(4′-Fluorophenyl)-3-hydroxymethyl-N-methyl-piperidine (Compound G)

Compound F of Example 7 (2.5 g), was dissolved in 25 mL of acetone and added directly to a solution of (−)-bis-p-toluoyl tartaric acid (5.6 g) dissolved in 25 mL acetone at ambient room temperature. The resulting acetone solution was stirred for about one hour at ambient room temperature and then at a cooled temperature in the range of about zero to about 5° C. for an additional 30 minutes.

A crystalline salt formed which was isolated by filtering through a Whatman #2 filter paper. The filtrate was concentrated to about 50% volume and then cooled as above. A second crop of crystalline salt was isolated as before. The first and second salt crops were combined and suspended in methylene chloride (50 mL) and aqueous 1N potassium hydroxide (50 mL) was added and the resulting mixture was agitated in a separatory funnel. The organic layer was then separated and dried over sodium sulfate, anhydrous, and evaporated under reduced pressure to yield a semi-solid material. The semi-solid material was triturated with hexane and produced a white crystalline powder, ([α]_{D}²⁶=−36° at a concentration of 5% in acetone), having a melting point in the range of about 100° to about 102° C. The recovered powder (1 g yield) had spectral characteristics (¹H-NMR and Mass Spectra data) that were consistent with the data of the compound produced in Example 7.

### EXAMPLE 9

### Large Scale Preparation of 4-Fluorocinnamonitrile (Compound B)

Powdered KOH (2.85 Kg) was suspended in degassed acetonitrile (20 L) solvent and mixed with stirring in a 70 L, 3-necked reactor equipped with a cooling jacket and an overhead mechanical stirrer. 4-Fluorobenzaldehyde (4 Kg) (Compound A) was dissolved in acetonitrile (2 L) solvent and the resulting solution was added to the stirring suspension of KOH at a rate such that the reaction temperature could be maintained below about 65° C. After the addition of Compound A was complete (about 5-10 minutes), the resulting reaction mixture was further stirred at the foregoing temperature for about 15 minutes. Subsequently, the reaction mixture was quenched by pouring a mixture of crushed ice and water (1:1, 18 L) into the reactor, an stirring was continued for an additional 30 minutes. The reaction mixture was then allowed to separate into two layers for 30 minutes. The lower aqueous layer was removed and discarded, and the upper organic layer was washed with brine (2×5 L), dried over anhydrous sodium sulfate (350 g). After filtration to remove the sodium sulfate, the organic layer was concentrated under reduced pressure to afford about 4 Kg of a thick oily product. The crude product was dissolved in ethylacetate (2 L) and applied to a bed of silica gel (2 Kg, 60 um). The product was eluted from the silica gel in three fractions with a 3:1 mixture of hexane/ethyl acetate (3.5 L×3 fractions). The combined fractions were concentrated under reduced pressure to afford Compound B as a pale yellow semi-solid (3.96 Kg, 83% yield). Analytical data on the product were substantially the same as the data provided in Example 1, except that the E/Z ratio was about 3, as determined by gas chromatography.

### EXAMPLE 10

### Large Scale Preparation of Diethyl[1-cyanomethyl-1-(4′-fluorophenyl)methyl]malonate (Compound C)

Sodium ethoxide (1.03 Kg) was added to 5 L of ethyl acetate, with stirring, in a 22 L three-necked reactor fitted with an overhead stirrer, addition funnel, thermocouple, a cooling bath and gas inlet/outlet connections. A solution of diethyl malonate (2.26 Kg) in ethyl acetate (2 L) was added to the suspension of sodium ethoxide over a period of about 45 minutes while stirring and maintaining the reaction temperature below about 20 C. A solution of Compound B from Example 9 (1.95 Kg) in ethyl acetate (2 L) was added to the malonate/ethoxide solution over about 30 minutes. The cooling bath was then replaced with a heating mantel and the reaction mixture was refluxed for about 4 hours (about 78° C.). The reaction was determined to be complete, based on analysis by Thin Layer Chromatography (TLC) (R_{f} values of 4-fluorocinnamonitrile=0.58; of diethylmalonate=0.54; and of reaction product=0.41), employing a solvent system of hexane and ethyl acetate (7:3), ultraviolet light and iodine vapor to expose the spots. Silica gel fluorescent plates were used. The reaction mixture was then cooled to ambient room temperature and quenched by the addition of glacial acetic acid (875 g) in water (5 L) and stirred for an additional 30 minutes. The aqueous phase was separated and discarded and the organic phase was washed with brine (3 L), dried over anhydrous sodium sulfate. After filtration to remove the sodium sulfate, the organic phase was concentrated in vacuo to afford the crude product as a yellow oil (4.26 Kg). The crude product was mixed with hexane (4 L), cooled to −12° C. and allowed to stand at that temperature overnight. Solid diethyl[1-cyanomethyl-1-(4′-fluorophenyl)methyl]malonate (Compound C) was isolated by vacuum filtration in 78% yield (3.16 Kg). Melting point was 38-43° C.

### EXAMPLE 11

### Large Scale Preparation of (±)-trans-3-Ethoxycarbonyl-4-(4′-fluorophenyl)piperidin-2-one (Compound D)

Compound C, produced by the method of Example 10 (3.63 Kg,) was dissolved in methanol (32 L) and to the solution was added activated Raney nickel (1 Kg, 50% wet) catalyst. The reaction mixture was evacuated and purged with nitrogen and then stirred under hydrogen atmosphere (H₂) at atmospheric pressure by continuous sparging of the reaction mixture with hydrogen, in a water bath set at a temperature of about 40° C., for a period of about 24 hours. The reaction mixture was then cooled to ambient room temperature and TLC analysis was performed as described in Example 2. TLC analysis showed that the reaction was complete (R_{f} values of starting material=0.41; of reaction product=0.12).

The cooled reaction mixture was allowed to stand at ambient room temperature to settle out the Raney nickel catalyst and the supernatant was filtered through Celite, and concentrated under reduced pressure to a white solid. This crude product was suspended in a 1:1 mixture of hexane and ethyl acetate (about 2-3 L) and the mixture was then cooled at −12° C. ovenight. Compound D ((±)-trans-3-ethoxycarbonyl-4-(4′-fluorophenyl)piperidin-2-one; 2.19 Kg, 70% yield) was recovered by filtration in the form of a crystalline white powder, having a melting point in the range of about 140° to about 145° C.

### EXAMPLE 12

### Large Scale Preparation of (±)-trans-4-(4′-Fluorophenyl)-3-hydroxymethylpiperidine (Compound E)

Aluminum hydride was generated in situ by dropwise addition of concentrated sulfuric acid (568 g, d=1.84) to a slurry of lithium aluminum hydride (LAH) (440 g) in anhydrous tetrahydrofuran (13 L) over a period of about 2-3 hours while concurrently cooling in an ice-water bath to maintain the temperature below 20° C. Compound D of Example 11, (1.12 Kg) was dissolved in anhydrous tetrahydrofuran (13.5 L) and then added slowly to the slurry of in situ-generated aluminum hydride over a period of about 1.5 hours. The reaction temperature was maintained below about 20° C. during the addition of Compound D. The reaction mixture was then heated at about 50° C. overnight under a nitrogen atmosphere. The reaction mixture was then cooled to about 5° C. and quenched by dropwise addition of 40% aqueous sodium hydroxide (475 g) and the reaction mixture was allowed to stir overnight until the slurry became white. The resultant slurry was filtered through Celite and the solid was washed with hot tetrahydrofuran (4×3 L). The combined filtrates were concentrated in vacuo to afford a sticky white solid, which was crystallized from ethyl acetate overnight at −12° C. The crystalline product was isolated by filtration to afford about 550 g (62% yield) of Compound E with a melting point of 123-124° C.

### EXAMPLE 13

### Large Scale Preparation of (±)-trans-4-(4′-fluorophenyl)-3-hydroxymethyl-N-methylpiperidine (Compound F)

Compound E, made by the method of Example 12 (3.63 Kg), was dissolved in methanol (26 L). Activated Raney nickel (1 Kg, 50% wet) catalyst and aqueous formaldehyde (formalin) solution (1.58 Kg, 37 wt %) were then added to the solution. The reaction mixture was stirred at a temperature of about 40° C. under hydrogen (H₂) at atmospheric pressure for about 12 hours. Stirring was stopped and the Raney nickel was allowed to settle. The supernatant was filtered through Celite and the filtrate was evaporated under reduced pressure to about ⅛th of its volume. Water (3 L) was added to the concentrated solution, which was allowed to stand at about −4° C. overnight. A white crystalline product was isolated from the aqueous methanol by filtration and labeled crop 1. The supernatant was concentrated to an oil in vacuo, and the oil solidified upon cooling to −4° C. overnight, this material was labeled crop 2. TLC analysis as decribed in Example 2 verified that both materials were substantially identical and analytical data were in agreement with those of Compound F from Example 7. The combined yield of both crops was 3.26 Kg (93%).

### EXAMPLE 14

### Large Scale Preparation of (−)-trans-4-(4′-Fluorophenyl)-3-hydroxymethyl-N-methyl-piperidine (Compound G)

Compound F of Example 13 (1.78 Kg), was dissolved in 30 L of acetone and stirred at ambient temperature. A solution of (−)-bis-p-toluoyl tartaric acid (4.56 Kg) dissolved in about 18 L of acetone was added to the solution of Compound F while cooling the reaction mixture. Stirring was continued overnight and a white precipitate formed. The precipitate was isolated by filtration to afford about 2.4 Kg of a white solid salt. The salt was dissolved in a mixture of dichlormethane (20 L) and IN aqueous potassium hydroxide (27 L). The mixture was stirred vigorously for about 30 minutes and then stirring was halted to allow the layers to separate. The organic layer was then collected and the aqueous layer was extracted with an additional 5 L of dichloromethane. The combined dichloromethane solutions were dried over anhydrous sodium sulfate, filtered and the supernatant was concentrated in vacuo to afford about 884 g of (−)-trans-4-(4′-Fluorophenyl)-3-hydroxymethyl-N-methyl-piperidine (Compound G, 96% yield). The analytical characteristics of the product were consistent with those obtained in Example 8.

### EXAMPLE 15

### Reductive Cyclization of Diethyl-2-cyano-3-(4′-fluorophenyl)glutarate

Diethyl-2-cyano-3-(4′-fluorophenyl)glutarate, prepared by the method of Wang et al. in European Patent Application EP 0802185 A1 (1.83 g), was dissolved in methanol (40 mL) and to the solution was added activated Raney nickel (400 mg, 50% wet) catalyst. The reaction mixture was evacuated and purged with nitrogen three times and then stirred under hydrogen atmosphere (H₂) at atmospheric pressure in a water bath set at a temperature of about 40° C. for a period of about 18 hours. The reaction mixture was then cooled to ambient room temperature. The cooled reaction mixture was filtered through Celite. The reaction flask and Celite residue were each washed with methanol (2×20 mL) and the filtrates combined. The combined filtrate was evaporated under reduced pressure to provide a white solid 3-ethoxycarbonyl-4-(4′-fluorophenyl)piperidin-6-one (mp 116-120° C.). NMR data matched those reported in EP 0802185 A1, and indicated that the product was a cis-trans mixture having a ratio of cis:trans of about 42:58. The cis/trans ratio was determined by integration of the methyl group resonance at 1.00 ppm for the trans isomer and 1.20 ppm for the cis isomer.

### EXAMPLE 16

### Reductive Cyclization of Diethyl-2-cyano-3-(4′-fluorophenyl)glutarate

The reaction of Example 15 was repeated, but at a hydrogen pressure of 2.5 atmospheres for 6 hours. The 3-ethoxycarbonyl-4-(4′-fluorophenyl)piperadine-6-one had a cis:trans ratio of 37:63 as determined by NMR analysis.

### EXAMPLE 17

### Reductive Cyclization of Diethyl-2-cyano-3-(4′-fluorophenyl)glutarate

The reaction of Example 16 was repeated, but at temeperature of about 60° C. for about 6 hours. The formed 3-ethoxycarbonyl-4-(4′-fluorophenyl)piperadin-6-one had a cis:trans ratio of 37:63 as determined by NMR analysis.

Examples 15-17 illustrate that diethyl-2-cyano-3-(4′-fluorophenyl)glutarate, a positional isomer of Compound C of Synthesis Scheme 2, disclosed by Wang et al., cyclizes with considerably lower stereo-control than does Compound C of the present invention. Upon reductive cyclization with Raney nickel, Compound C afford the 2-keto piperadine derivative Compound D having a substantially trans configuration. In contrast, the reductive cyclization of the Wang et al. positional isomer of Compound C, under similar conditions to those of the present invention, affords the 6-keto piperidine intermediate 3-ethoxycarbonyl-4-(4′-fluorophenyl)piperidin-6-one with trans:cis ratios of about 58:42 to about 63:37, thus necessitating an additional process step to either remove the cis isomer or convert the cis product to the desired trans configuration.

We claim:

1. A process for synthesis of a racemic mixture of compounds having structural formula (I): 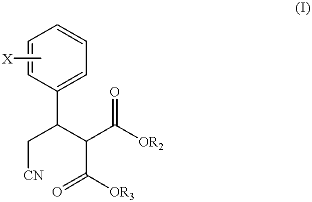
where X is halo, C₁-C₁₀ alkoxy, C₁-C₁₀ haloalkyl, or hydroxy; and R₂ and R₃ are each C₁-C₄ alkyl and R₂ and R₃ are the same; which process comprises condensing a cinnamonitrile represented by the structural formula: 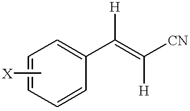
wherein X is as defined in structural formula (I), with a diester malonate represented by the structural formula: 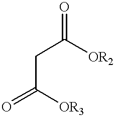
wherein each of R₂ and R₃ is as defined in structural formula (I).

2. The process of claim 1 wherein X is halo and R₂ and R₃ are each ethyl.

3. The process of claim 2, wherein X is fluoro.

4. The process of claim 1 wherein the cinnamonitrile is 4-fluorocinnamonitrile and the diester malonate is diethyl malonate.

5. The process of claim 1 further including the step of recovering the racemic compound of structural formula (I).

6. A product of the process of claim 1 having structural formula (I).

7. A racemic mixture of compounds having structural formula (I): 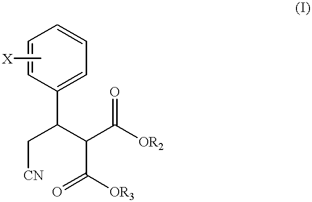
where X is halo, C₁-C₁₀ alkoxy, C₁-C₁₀ haloalkyl, or hydroxy; and R₂ and R₃ are each C₁-C₄ alkyl, and R₂ and R₃ are the same.

8. The racemic mixture of compounds of claim 7 wherein X is fluoro and R₂ and R₃ are each ethyl.

9. Racemic diethyl[1-cyanomethyl-1(4′-fluorophenyl)methyl]malonate characterized by a melting point in the range of about 35° to about 50° C.

10. A process for synthesis of a racemic mixture of compounds having structural formula (II): 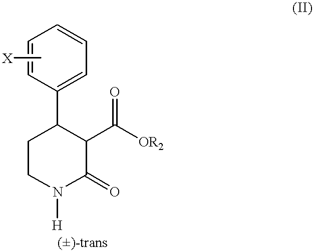
where X is halo, C₁-C₁₀ alkoxy, C₁-C₁₀ haloalkyl, or hydroxy; and R₂ is C₁-C₄ alkyl; which process comprises reducing the compound of claim 1 with hydrogen and a catalyst.

11. The process of claim 10 wherein X is a halo, and R₂ is ethyl.

12. The process of claim 11 wherein X is fluoro.

13. The process of claim 10 further including the step of recovering the racemic compound of structural formula (II).

14. The process of claim 10 wherein the catalyst is Raney-nickel.

15. The process of claim 10 further comprising the step of reducing the compound of structural formula (II) to racemic (±)-trans arylpiperidine carbinol.

16. The process of claim 15 wherein the reduction is accomplished by treatment of a compound of structural formula (II) with a metal hydride.

17. The process of claim 16 wherein the metal hydride is lithium aluminum hydride or aluminum hydride.

18. The process of claim 17 wherein the aluminum hydride is generated in situ by reaction of lithium aluminum hydride with a mineral acid.

19. The process of claim 18 wherein the mineral acid is sulfuric acid.

20. The process of claim 15 further comprising the step of alkylating the (±)-arylpiperidine carbinol to racemic (±)-trans N-substituted arylpiperidine carbinol.

21. The process of claim 20 further comprising the step of isolating substantially enantiomerically pure (−)-trans- arylpiperidine carbinol from the racemic (±)-trans-N-substituted arylpiperidine carbinol.

22. The process of claim 21 wherein the isolating step comprises adding a chiral acid of (−) optical characteristic to form a diasteriomeric salt in an organic solvent, crystallizing one diastereomer of the salt, isolating the crystalline salt and neutralizing the isolated salt with an aqueous base to provide substantially optically pure (−)-trans configured arylpiperidine carbinol.

23. A product of the process of claim 10 having structural formula (II).

24. A racemic mixture of compounds having structural formula (II): 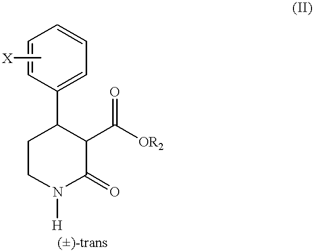
where X is halo, C₁-C₁₀ alkoxy, C₁-C₁₀ haloalkyl,, or hydroxy; and R₂ is C₁-C₄ alkyl.

25. The racemic mixture of compounds of claim 24 wherein X is fluoro and R₂ is ethyl.

26. The racemic mixture of compounds of claim 25 having greater than 90% trans configuration.

27. Racemic (±)-trans-3-ethoxycarbonyl-4-(4′-fluorophenyl) piperidin-2-one characterized by a melting point in the range of about 140° to about 150° C.
